(19) United States
(12) Patent Application Publication
(10) Pub. No.: US 2009/0035363 A1
(43) Pub. Date: Feb. 5, 2009

(54) Photoactive Compounds and Compositions and Uses Thereof

(76) Inventors: Raghavan Rajagopalan, Solon, OH (US); Richard B. Dorshow, St. Louis, MO (US)

Correspondence Address: Mallinckrodt Inc., 675 McDonnell Boulevard, HAZELWOOD, MO, 63042, US

(21) Appl. No.: 12/281,338

(22) PCT Filed: Mar. 9, 2007
(86) PCT No.: PCT/US07/06211

§ 371 Date: Sep. 2, 2008

Related U.S. Application Data
(60) Provisional application No. 60/781,530, filed on Mar. 10, 2006.

(51) Int. Cl. A61K 9/127; A61K 38/08; A61K 9/12

(52) U.S. Cl. 424/450; 514/16; 424/45

(57) ABSTRACT

Photoactive compounds and compositions, as well as methods of using the same. For example, compositions of the invention may be used in Type 1 phototherapy, Type 2 phototherapy, or a combination of Types 1 and 2 phototherapy.

### FIELD OF THE INVENTION

This invention relates generally to photoactive compounds and compositions and their use in photochemical procedures (e.g., medical phototherapeutic procedures).

### BACKGROUND

The use of visible and near-infrared (NIR) light in clinical practice is growing rapidly. Compounds absorbing or emitting in the visible, NIR, or long-wavelength (UV-A, >350 nm) region of the electromagnetic spectrum are potentially useful for optical tomographic imaging, endoscopic visualization, and phototherapy. However, a major advantage of biomedical optics lies in its therapeutic potential. Phototherapy has been demonstrated to be a safe and effective procedure for the treatment of various surface lesions, both external and internal. Its efficacy is comparable to that of radiotherapy, but without the harmful radiotoxicity to critical non-target organs.

Phototherapy has been in existence for many centuries and has been used to treat various skin surface ailments. As early as 1400 B.C. in India, plant extracts (psoralens), in combination with sunlight, were used to treat vitiligo. In 1903, Von Tappeiner and Jesionek used eosin as a photosensitizer for the treatment of skin cancer, lupus of the skin, and condylomata of female genitalia. Over the years, the combination of psoralens and ultraviolet A (low-energy) radiation has been used to treat a wide variety of dermatological diseases including psoriasis, parapsoriasis, cutaneous T-cell lymphoma, eczema, vitiligo, greata, and neonatal bilirubinemia. Although the potential of cancer phototherapy has been recognized since early 1900's, systematic studies to demonstrate safety and efficacy began only in 1967 with the treatment of breast carcinoma. Dougherty et al. subsequently conclusively established that long-term cure is possible with photodynamic therapy (PDT). Currently, phototherapeutic methods are also being investigated for the treatment of some cardiovascular disorders such as atherosclerosis and vascular restenosis, for the treatment rheumatoid arthritis, and for the treatment of some inflammatory diseases such as Crohn's disease.

Phototherapeutic procedures require photosensitizers that have high absorptivity. These compounds should preferably be chemically inert, and become activated only upon irradiation with light of an appropriate wavelength. Light-initiated selective tissue injury can be induced when these photosensitizers bind to target tissues, either directly or through attachment to a bioactive carrier. Furthermore, if the photosensitizer is also a chemotherapeutic agent (e.g. anthracycline antitumor agents), then an enhanced therapeutic effect can be attained.

Effective photochemical agents should have the following properties: (a) large molar extinction coefficient; (b) long triplet lifetime; (c) high yield of singlet oxygen and/or other reactive intermediates, viz., free radicals, nitrenes, carbenes, open-shell ionic species such as cabonium ions and the like; (d) efficient energy or electron transfer to cellular components; (e) low tendency to form aggregation in aqueous milieu; (4) efficient and selective targeting of lesions; (g) rapid clearance from blood and non-target tissues; (h) low systemic toxicity; and (i) lack of mutagenicity.

Photosensitizers operate via two distinct pathways, termed Types 1 and 2. The type 1 mechanism is shown in the following scheme:

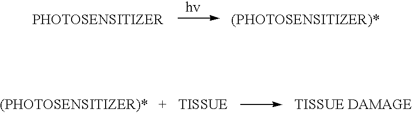

After photoexcitation, the Type 1 mechanism involves direct energy or electron transfer from the photosensitizer to the cellular components, thereby causing cell death. After photoexcitation, the Type 2 mechanism involves distinct steps as shown in the following scheme:

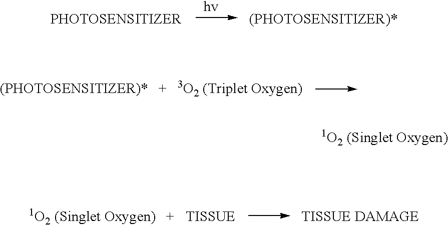

In the first step, singlet oxygen is generated by energy transfer from the triplet excited state of the photosensitizer to the oxygen molecules surrounding the tissues. In the second step, collision of a singlet oxygen with the tissues promotes tissue damage. In both Type 1 and Type 2 mechanisms, the photoreaction proceeds via the lowest triplet state of the photosensitizer. Hence, a relatively long triplet lifetime is required for effective phototherapy. In contrast, for diagnostic imaging purposes, a relatively short triplet lifetime is required to avoid photodamage to the tissue caused by photosensitizers.

The biological basis of tissue injury brought about by tumor phototherapeutic agents has been the subject of intensive study. Various reasonable biochemical mechanisms for tissue damage have been postulated even though the type and number of photosensitizers employed in these studies are relatively small. These biochemical mechanisms are as follows: a) cancer cells upregulate the expression of low density lipoprotein (LDL) receptors, and PDT agents bind to LDL and albumin selectively; (b) porphyrin-like substances are selectively taken up by proliferative neovasculature; (c) tumors often contain an increased number of lipid bodies and are thus able to bind to hydrophobic photosensitizers; (d) a combination of “leaky” tumor vasculature and reduced lymphatic drainage causes porphyrin accumulation; (e) tumor cells may have increased capabilities for phagocytosis or pinocytosis of porphyrin aggregates; (f) tumor associated macrophages may be largely responsible for the concentration of photosensitizers in tumors; and (g) cancer cells may undergo apoptosis induced by photosensitizers. Among these mechanisms, (f) and (g) are the most general and, of these two alternatives, there is a general consensus that (f) is the most likely mechanism by which the phototherapeutic effect of porphyrin-like compounds is induced.

Most of the currently known photosensitizers are commonly referred to as PDT agents and operate via the Type 2 mechanism. For example, Photofrin II, a hematoporphyrin derivative, was approved by the United States Food and Drug Administration for the treatment of bladder, esophageal, and late-stage lung cancers. However, Photofrin II has been shown to have several drawbacks: low molar absorptivity, (ε=3000M⁻¹), low singlet oxygen quantum yield (N=0.1), chemical heterogeneity, aggregation, and prolonged cutaneous photosensitivity. Hence, there has been considerable effort in developing safer and more effective photosensifizers for PDT that exhibit improved light absorbance properties, better clearance, and decreased skin photosensitivity compared to those of Photofrin II. These photosensitizers include monomeric porphyrin derivatives, corrins, cyanines, phthalocyanines, phenothiazines, rhodamines, hypocrellins, and the like. However, these phototherapeutic agents also mainly operate via the Type 2 mechanism.

Surprisingly, there has not been much attention directed at developing Type 1 phototherapeutic agents, despite the fact that the Type 1 mechanism seems inherently more efficient than the Type 2 mechanism. First, unlike Type 2, Type 1 photosensitizers do not require oxygen for causing cellular injury. Second, the Type 1 mechanism involves two steps (photoexcitation and direct energy transfer) whereas the Type 2 mechanism involves three steps (photoexcitation, singlet oxygen generation, and energy transfer). Furthermore, some tumors have hypoxic regions that render the Type 2 mechanism ineffective. In spite of the drawbacks associated with the Type 2 mechanism, however, only a small number of compounds have been developed that operate through the Type 1 mechanism, e.g. anthracyline antitumor agents.

Thus, there is a need to develop effective phototherapeutic agents that operate through the Type 1 mechanism. Phototherapeutic efficacy can be further enhanced if the excited state photosensifizers can generate reactive intermediates such as free radicals, nitrenes, carbenes, and the like. These have much longer lifetimes than the excited chromophore and have been shown to cause considerable cell injury.

### SUMMARY

The present invention discloses novel organic compounds and compositions that may be utilized in photochemical procedures. A photochemical procedure encompasses both medical therapeutic and diagnostic procedures, as will be subsequently described.

A first aspect of the invention is directed to a compound having the general formula E1-L-Ar—X—PA, where Ar is a photosensifizer, PA is a photoactive compound, and each of E1, L, and X is optional.

The photosensitizer (Ar) is a chromophore that generally contains large cyclic or aromatic rings. The photosensitizer may be linked either directly or indirectly to E1, which in some embodiments can be selected to target the compound to a specific site, or which in other embodiments can be hydrogen. The photosensitizer (Ar) is linked directly or indirectly to a photoactive compound (PA) that, when photoactivated, additionally damages tissues via a Type 1 or Type 2 mechanism. It will be appreciated that, by selecting specific components for E1, one can target the compound to reach a specific body site, for example, a tumor site where photoactivation will destroy tumor cells. It will also be appreciated that a linker L, if present, can be selected to appropriately link E1 to the photosensitizer (Ar). For instance, in some embodiments, it may be desirable to select a linker (L) that will provide a desired amount of space between E1 and a bulky aromatic or cyclic photosensitizer.

PA is a photoactive compound such as an azide, diazoalkane, peroxide, alkyliodide, sulfenate, azidoalkyl, azidoaryl, diazoalkyl, diazoaryl, peroxoalkyl, peroxoaryl, iodoalkyl, azoalkyl, cyclic or acyclic azoalkyl, sulfenatoalkyl, sulfenatoaryl, etc. that produce nitrenes, free radicals, carbenes, etc. upon photoactivation.

Numerous combinations of Ar and PA are possible to provide Type 1 phototherapy, as will be described. Additionally, it will be appreciated that many formulations are possible because of the various linkers and targeting moieties that may be used, as will also be described.

Ar is a photosensitizer including at least one substituent represented by any of formulas I-VIII

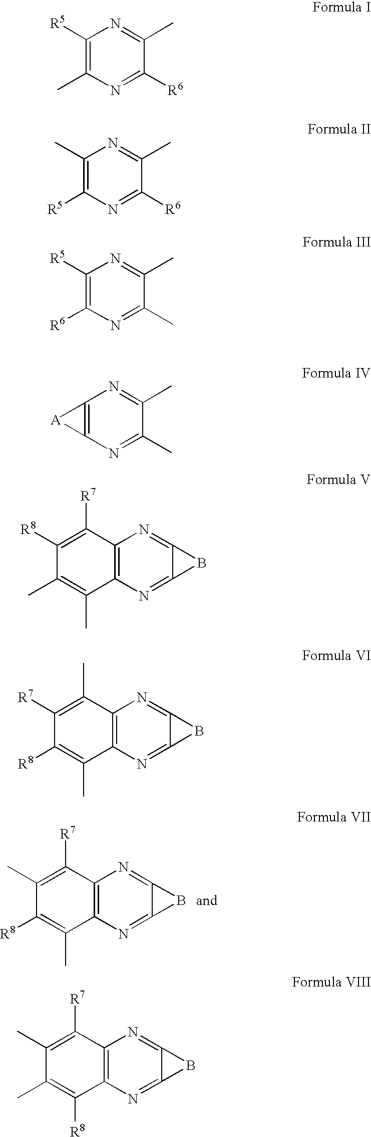

E1, if present, may be hydrogen or a targeting moiety. For instance, in some embodiments, E1 may be a receptor binding molecule, such as a whole or fragmented somatostatin receptor binding molecule, whole or fragmented ST receptor binding molecule, whole or fragmented neurotensin receptor binding molecule, whole or fragmented bombesin receptor binding molecule, whole or fragmented cholecystekinin (CCK) receptor binding molecule, whole or fragmented steroid receptor binding molecule, or whole or fragmented carbohydrate receptor binding molecule.

X, if present, is a linker between the photosensitizer (Ar) and the photoactive compound (PA) and may be selected from a single bond, —(CH₂)ₐ—, —CO—, —OCO—, —HNCO—, —(CH₂)ₐCO—, —(CH₂)ₐOCO—, C₁-C₁₀ alkyl, C₅-C₁₀ aryl, C₅-C₁₀ heteroaryl, C₁-C₁₀ acyl, nitro, cyano, —(CH₂)ₐCO₂—, —(CH₂)ₐNR¹—, —NR¹CO—, —(CH₂)ₐCONR¹—, —(CH₂)ₐ—SO—, —(CH₂)ₐSO₂—, —(CH₂)ₐCON(R¹)—, —(CH₂)ₐN(R¹)CO—, —(CH₂)ₐN(R¹)CON(R²)— and —(CH₂)ₐN(R¹)CSN(R²)—.

L, if present, is a linker between the photosensitizer (Ar) and E1 and may be selected from a single bond, —HNCO—, —CONR³, —(CH₂)_{b}—, —(CH₂)_{b}CONR³—, —N(R³)CO(CH₂)_{b}—, —OCO(CH₂)_{b}—, —(CH₂)_{b}CO₂—, —OCONH—, —OCO₂—, —HNCONH—, —HNCSNH—, —HNNHCO—, —OSO₂—, —NR³(CH₂)_{b}CONR⁴—, —CONR³(CH₂)_{b}NR⁴CO—, —NR³CO(CH₂)_{b}CONR⁴—, —(CH₂)_{b}CON(R³)—, —(CH₂)_{b}N(R³)CO—, —(CH₂)_{b}N(R³)CON(R⁴)— and —(CH₂)_{b}N(R³)CSN(R⁴)—.

In the above structures, each of R¹ to R⁴ may independently be selected from hydrogen, C1-C10 alkyl, —OH, C5-C10 aryl, C1-C10 hydroxyalky, C1-C10 polyhydroxyalkyl, C1-C10 alkoxyl, C1-C10 alkoxyalkyl, —SO₃H, —(CH₂)_{c}CO₂H and —(CH₂)_{c}NR⁹R¹⁰.

Each of R⁹ and R¹⁰ may independently be selected from hydrogen, C1-C10 alkyl, C5-C10 aryl and C1-C10 polyhydroxyalkyl.

Each of a, b, and c may independently range from 0 to 10.

Each of A and B may independently be selected from —(CH₂)_{d}Y(CH₂)ₑ—, —C(R¹¹)═C(R¹²)—C(R¹³)═C(R¹⁴)—, —N═C(R¹²)—C(R¹³)═C(R¹⁴)—, —C(R¹¹)═N—C(R¹³)═C(R¹⁴)—, —C(R¹¹)═C(R¹²)—N═C(R¹⁴)—, —C(R¹¹)═C(R¹²)C(R¹³)═N—, —C(R¹¹)═C(R¹²)—N(R¹⁵)—, —C(R¹¹)═C(R¹²)—O—, C(R¹¹)═C(R¹²)—S—, —N═C(R¹¹)—N(R¹⁵)—, —N═C(R¹¹)—O—, —N═C(R¹¹)—S—, —C(R¹¹)═N—N(R¹⁵)—, —C(R¹¹)═N—N(R¹⁵)—, —C(R¹¹)═N—O—, —N═N—N(R¹⁵)— and —N═N—O— or —N═N—S—;

Y may be selected from —O—, —NR¹⁶—, —SO— or —SO₂—.

Each of d and e may independently vary from 0 to 3.

R¹⁶ may be selected from hydrogen, C₁-C₁₀ alkyl, C₅-C₁₀ aryl, C₁-C₁₀ hydroxyalkyl, and C₁-C₁₀ alkoxyalkyl.

Each of R⁵ to R⁸ and each of R¹¹ to R¹⁵ may independently be selected from hydrogen, C₁-C₁₀ alkyl, C₅-C₁₀ aryl, C₁-C₁₀ hydroxyalkyl, C₁-C₁₀ alkoxyalkyl, C₅-C₁₀ heteroaryl, C₁-C₁₀ acyl, nitro, cyano, —(CH₂)_{f}N₃, —(CH₂)_{f}CO₂R¹⁶, —(CH₂)_{f}NR¹⁶R¹⁷, —NR¹⁶CON₃, —(CH₂)_{f}CONR¹⁶R¹⁷, —(CH₂)_{f}CON₃, —(CH₂)_{f}SON₃, —(CH₂)_{f}SO₂N₃, —(CH₂)_{f}CON(R¹⁶)E2, —(CH₂)_{f}N(R¹⁶)COE2, —(CH₂)_{f}N(R¹⁷)CON(R¹⁷)E2 and —(CH₂)ᵣN(R¹⁶)CSN(R¹⁷)E2.

f may vary from 0 to 10.

Each of R¹⁶ and R¹⁷ may be independently selected from hydrogen, C₁-C₁₀ alkyl, C₅-C₁₀ aryl, C₁-C₁₀ hydroxyalkyl and C₁-C₁₀ alkoxyalkyl.

Each of E1 and E2 may independently be hydrogen or a targeting moiety.

In some embodiments, E1 and E2, if present, are each independently a whole or fragmented somatostatin receptor binding molecule, whole or fragmented ST receptor binding molecule, whole or fragmented neurotensin receptor binding molecule, whole or fragmented bombesin receptor binding molecule, whole or fragmented CCK receptor binding molecule, whole or fragmented steroid receptor binding molecule, and whole or fragmented carbohydrate receptor binding molecule. In some embodiments, E1 and E2 are both receptor binding molecules of the same type. For instance, in some embodiments, E1 and E2 are both a whole or fragmented somatostatin receptor binding molecule, whole or fragmented ST receptor binding molecule, whole or fragmented neurotensin receptor binding molecule, whole or fragmented bombesin receptor binding molecule, whole or fragmented CCK receptor binding molecule, whole or fragmented steroid receptor binding molecule, and whole or fragmented carbohydrate receptor binding molecule. In some embodiments, E1 may be a receptor binding molecule of a first type, and E2 may be a receptor binding molecule of a second type different from E1.

For targeting purposes, external attachment of a targeting moiety may be used. If photoactive compounds and/or photosensitizers themselves preferentially accumulate in a target tissue, however, such a targeting moiety may not be needed. For example, if Ar is an anthracycline moiety, it may tend to bind to cancer cells directly and not require a targeting moiety. Thus, E1 may be absent or may be hydrogen. A targeting moiety includes but is not limited to one or more specific sites of a molecule which will bind to a particular complementary site, such as the specific sequence of amino acids in a region of an antibody that binds to the specific antigen binding site. A targeting moiety is not limited to a particular sequence or site, but includes anything that will target an inventive compound and/or composition to a particular anatomical and/or physiological site. Examples of compounds that may be used as targeting moieties include, but are not limited to, whole receptor binding compounds or fragments of receptor binding compounds.

A second aspect of the present invention is directed to a biocompatible composition including at least one biocompatible excipient (e.g., a buffer, emulsifier, surfactant, electrolyte, or combination thereof) and a compound having the general formula E1-L-Ar—X—PA as described herein.

In some embodiments of this second aspect, a liposome may be utilized as a carrier or vehicle for the composition. For example, in some embodiments, the photosensitizer may be a part of the lipophilic bilayers, and the targeting moiety, if present, may be on the external surface of the liposome. As another example, a targeting moiety may be externally attached to the liposome after formulation for targeting the liposome (which contains the inventive compound) to the desired tissue, organ, or other site in the body.

Still a third aspect of the invention is directed to a method of using a compound of the general formula E1-L-Ar—X—PA described herein. In this method, an effective amount of the compound (e.g., as a component of a biocompatible composition) is administered to a target tissue in an animal. The target tissue is then exposed to light sufficient to activate the compound. The compound may be allowed to accumulate in the target tissue before the target tissue is exposed to light (e.g., light having a wavelength between about 300 and 950 nm). In some embodiments, the compound may be used in a phototherapeutic procedure in which the target tissue is exposed to light of sufficient power and fluence rate to photoactivate the compound and perform phototherapy. Incidentally, photoexcitation of the aromatic photosensitizers of formulas I-VIII effects a rapid intramolecular energy transfer to PA, resulting in bond rupture and production of nitrene and nitrogen gas. The nitrogen that is released is in a vibrationally excited state, which may cause additional cellular injury.

These and other embodiments of the inventive compounds, compositions, and methods will be apparent in light of the following figures, description, and examples.

### BRIEF DESCRIPTION OF THE FIGURES

FIG. 1a is a general Type 1 photoactivation scheme.

FIG. 1b is a general Type 2 photoactivation scheme.

FIG. 2a is a photoactivation scheme showing formation of diradicals.

FIG. 2b is a photoactivation scheme showing formation of singlet oxygen.

FIG. 3 is a bioconjugation scheme of the invention.

### DETAILED DESCRIPTION

The invention discloses novel organic compounds, compositions, and photochemical procedures. A photochemical procedure encompasses any type of biologic procedure using the inventive compounds, and includes in vivo and in vitro procedures, and therapeutic and diagnostic procedures. The following is a detailed description of various embodiments of exemplary compounds of the general formula E1-L-Ar—X—PA.

PA is a photoactive compound that includes an azide, diazoalkane, peroxide, alkyliodide, sulfenate, azidoalkyl, azidoaryl, diazoalkyl, diazoaryl, peroxoalkyl, peroxoaryl, iodoalkyl, azoalkyl, cyclic and/or acyclic azoalkyl, sulfenatoalkyl, or sulfenatoaryl.

Ar is a photosensitizer that is an aromatic or a heteroaromatic chromophore containing at least one of formulas I-VIII

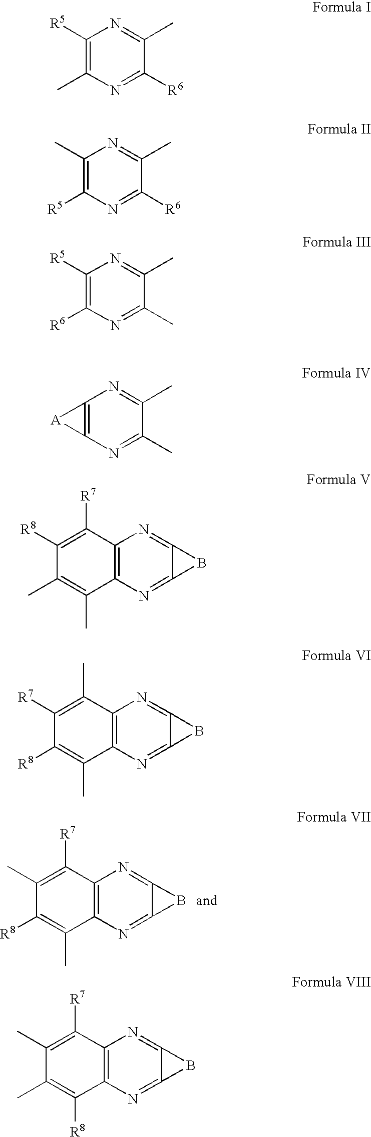

E1, if present, is either hydrogen or a targeting moiety. Again, a targeting moiety generally refers to a particular region of the compound that is recognized by, and binds to, a target cell, tissue, organ, etc. A targeting moiety may include an antibody (all or a portion, and monoclonal or polyclonal), peptide, pepbdomimetic, carbohydrate, glycomimetic, drug, hormone, nucleic acid, lipid, albumin, receptor binding molecule, inclusion compound (a compound that has a cavity with a defined volume such that it can incorporate small molecules or a part of a small molecule) such as cyclodextrins (cyclodextrins can accommodate hydrophobic residues such as adamantine, benzene, etc), etc.

Targeting moieties may be part of a biomolecule which include hormones, amino acids, peptides, peptidomimetics, proteins, nucleosides, nucleotides, nucleic acids, enzymes, carbohydrates, glycomimetics, lipids, albumins, mono- and polyclonal antibodies, receptors, inclusion compounds such as cyclodextrins, and receptor binding molecules. Specific examples of targeting moieties include steroid hormones for the treatment of breast and prostate lesions, whole or fragmented somatostatin, bombesin, and neurotensin receptor binding molecules for the treatment of neuroendocrine tumors, whole or fragmented cholecystekinin receptor binding molecules for the treatment of lung cancer, whole or fragmented heat sensitive bacterioendotoxin (ST) receptor and carcinoembryonic antigen (CEA) binding molecules for the treatment of colorectal cancer, dihyroxyindolecarboxylic acid and other melanin producing biosynthetic intermediates for melanoma, whole or fragmented integrin receptor and atherosclerotic plaque binding molecules for the treatment of vascular diseases, and whole or fragmented amyloid plaque binding molecules for the treatment of brain lesions. In some embodiments, E1, if present, is selected from octreotide and octreotate peptides, heat-sensitive bacterioendotoxin receptor binding peptide, carcinoembryonic antigen antibody (anti-CEA), bombesin receptor binding peptide, neurotensin receptor binding peptide, cholecystekinin receptor binding peptide, or estrogen.

As a non-limiting example, and with respect to compounds that may be used as E1 because they bind to a receptor, one skilled in the art would appreciate that diethylstilbesterol is not a steroid but strongly binds to the estrogen receptor (a steroid receptor); testosterone does not bind to the estrogen receptor, testosterone and esterone do not bind to the corticosteroid receptors, cortisone and aldosterone do not bind to the sex hormone receptors, and the following compounds are known to bind to the estrogen receptor, namely, estratriol, 17β-aminoestrogen (AE) derivatives such as prolame and butolame, drugs such as tamoxifen, ICI-164384, raloxifene, genistein, 17β-estradiol, glucocorticoids, progesterone, estrogens, retinoids, fatty acid derivatives, phytoestrogens, etc. Thus, one skilled in the art would know how to select compounds to target and/or to avoid a particular site.

For targeting purposes, an external attachment of a targeting moiety is usually desirable unless the compounds themselves preferentially accumulate in the target tissue, thereby obviating the need for an additional binding group. For example, administering delta-aminolevulinic acid, an intermediate in porphyrin biosynthesis, results in a two-fold uptake of porphyrins in tumors compared to normal tissues. Similarly, administering dihydroxyindole-2-carboxylic acid, an intermediate in melanin biosynthesis, produces substantially enhanced levels of melanin in melanoma cells compared to normal cells. Thus, an inventive compound may be delivered to the site of a lesion by attaching it to these types of biosynthetic intermediates. Although this targeting is less specific than in embodiments where a specific targeting moiety is included in the compound, it still targets the compound to a desired site and thus is another embodiment of the invention.

X, if present, is a linker between the photosensitizer (Ar) and the photoactive compound (PA) and is selected from a single bond, —(CH₂)ₐ—, —CO—, —OCO—, —HNCO—, —(CH₂ₐCO—, —(CH₂)ₐOCO—, C₁-C₁₀ alkyl, C₅-C₁₀ aryl, C₅-C₁₀ heteroaryl, C₁-C₁₀ acyl, nitro, cyano, —(CH₂)ₐCO₂—, —(CH₂)ₐNR¹—, —NR¹CO—, —(CH₂)ₐCONR¹—, —(CH₂)ₐSO—, —(CH₂)ₐSO₂—, —(CH₂)ₐCON(R¹)—, —(CH₂)ₐN(R¹)CO—, —(CH₂)ₐN(R¹)CON(R²)— and —(CH₂)ₐN(R¹)CSN(R²)—.

L, if present, is a linker between the photosensitizer and E1 and is selected from a single bond, —HNCO—, —CONR³, —(CH₂)_{b}—, —(CH₂)_{b}CONR³—, —N(R³)CO(CH₂)_{b}—, —OCO(CH₂)_{b}—, —(CH₂)_{b}CO₂—, —OCONH—, —OCO₂—, —HNCONH—, —HNCSNH—, —HNNHCO—, —OSO₂—, —NR³(CH₂)_{b}CONR⁴—, —CONR³(CH₂)_{b}NR⁴CO—, —NR³CO(CH₂)_{b}CONR⁴—, —(CH₂)_{b}CON(R³)—, —(CH₂)_{b}N(R³)CO—, —(CH₂)_{b}N(R³)CON(R⁴)— and —(CH₂)_{b}N(R³)CSN(R⁴)—.

Each of R¹ to R⁴ is independently selected from hydrogen, C1-C10 alkyl, —OH, C5-C10 aryl, C1-C10 hydroxyalky, C1-C10 polyhydroxyalkyl, C1-C10 alkoxyl, C1-C10 alkoxyalkyl, —SO₃H, —(CH₂)_{c}CO₂H, and —(CH₂)_{c}NR⁹R¹⁰.

Each R⁹ and R¹⁰ is independently selected from hydrogen, C1-C10 alkyl, C5-C10 aryl, and C1-C10 polyhydroxyalkyl.

Each of a, b, and c independently ranges from 0 to 10.

Each of A and B is independently selected from —(CH₂)_{d}Y(CH₂)ₑ—, —C(R¹¹)═C(R¹²)—(R¹³)═C(R¹⁴)—, —N═C(R¹²)—C(R¹³)═C(R¹⁴)—, —(R¹¹)═N—(R¹³)═C(R¹⁴)—, —(R¹¹)═C(R¹²)—N═C(R¹⁴)—, C(R¹¹)═C(R¹²)—C(R¹³)═N—, —C(R¹¹)═C(R¹²)—N(R¹⁵)—, —C(R¹¹)═C(R¹²)—O—, —C(R¹¹)═C(R¹²)—S—, —N═C(R¹¹)—N(R¹⁵)—, —N═C(R¹¹)—O—, —N═C(R¹¹)—S—, —C(R¹¹)═N—N(R¹⁵)—, —C(R¹¹)═N—N(R¹⁵)—, C(R¹¹)═N—O—, —N═N—N(R¹⁵)— and —N═N—O— or —N═N—S—.

Y is selected from —O—, —NR¹⁶—, —S—, —SO— and —SO₂—.

Each of d and e independently vary from 0 to 3.

R¹⁶ is selected from hydrogen, C₁-C₁₀ alkyl, C₅-C₁₀ aryl, C₁-C₁₀ hydroxyalkyl, or C₁-C₁₀ alkoxyalkyl.

Each of R⁵ to R⁸ and each of R¹¹ to R¹⁵ is independently selected from hydrogen, C₁-C₁₀ alkyl, C₅-C₁₀ aryl, C₁-C₁₀ hydroxyalkyl, C₁-C₁₀ alkoxyalkyl, C₅-C₁₀ heteroaryl, C₁-C₁₀ acyl, nitro, cyano, —(CH₂)_{f}N₃, —(CH₂)_{f}CO₂R¹⁶, —(CH₂)_{f}NR¹⁶R¹⁷, —NR¹⁶CON₃, —(CH₂)_{f}CONR¹⁶R¹⁷, —(CH₂)_{f}CON₃, —(CH₂)_{f}SON₃, —(CH₂)_{f}SO₂N₃, —(CH₂)_{f}CON(R¹⁶)E2, —(CH₂)_{f}N(R¹⁸)COE2, —(CH₂)_{f}N(R¹⁶)CON(R¹⁷)E2 and —(CH₂)_{f}N(R¹⁶)CSN(R¹⁷)E2.

f varies from 0 to 10.

Each of R¹⁶ and R¹⁷ is independently selected from hydrogen, C₁-C₁₀ alkyl, C₅-C₁₀ aryl, C₁-C₁₀ hydroxyalkyl and C₁-C₁₀ alkoxyalkyl.

E2 is defined in the same manner as E1, and each occurrence of E1 and E2 is independently hydrogen or a targeting moiety.

Compounds of the invention may be used in compositions and in vitro or in vivo biological procedures. Conjugation of a small molecule to a small peptide or other small molecule carrier generally preserves receptor binding capability. Coupling of diagnostic and radiotherapeutic agents to biomolecules can be accomplished by methods well known in the art, as disclosed in Hnatowich et al., Radiolabeling of Antibodies: A simple and efficient method. Science, 1983, 220, 613; A. Pelegrin et al., Photoimmunodiagnostics with antibody-fluorescein conjugates: in vitro and in vivo preclinical studies. Journal of Cellular Pharmacology, 1992, 3, 141-145, and U.S. Pat. No. 5,714,342, which are expressly incorporated by reference herein in their entirety.

Formulas I-VIII are members of a class of small molecules that possess desirable absorption and emission properties in the UV-A, visible and NIR region of the electromagnetic spectrum. Various substituents such as electron donating groups, electron withdrawing groups, lipophilic groups, or hydrophilic groups can be attached at the respective carbon atoms for altering physicochemical and/or biological properties, as known to one skilled in the art. The substituents may also optionally include E2 (which is either hydrogen or a targeting moiety) that will selectively bind to a desired target tissue or lesion. The target may be a biological receptor, an enzyme, etc.

In some embodiments, at least the photosentizer (Ar) of the compound operates through a Type 1 photoactive mechanism capable of generating reactive intermediates such as free radicals, nitrenes, carbenes, and the like that can result in injury or death to cells when the photochemically active compound is at a target site such as a tumor or lesion. Compounds of the invention absorb radiation in the low-energy, ultraviolet, visible, or NIR region of the electromagnetic spectrum, and are useful for photodiagnosis, phototherapy, etc. of tumors and other lesions. In some embodiments, the photosensitizer (Ar) portion of the compound may be tuned (e.g., via substitution of the π system) to customize electronic and/or optical properties of the photosensitizer. For instance, it may be desirable to tune a photosensitizer so that it absorbs in the visible red region of the spectrum and operates through a Type 2 photoactive mechanism.

As previously described, Type 1 agents contain a labile precursor that undergoes photofragmentation upon direct irradiation with light of a desired wavelength, and produce reactive intermediates such as nitrenes, carbenes, or free radicals from photoactive compounds (PA). PA may be azides, diazoalkanes, peroxides, alkyliodides, sulfenates, azidoalkyl, azidoaryt, diazoalkyl, diazoaryl, peroxoalkyl, peroxoaryl, iodoalkyl, azoalkyl, cyclic or acyclic azoalkyl, sulfenatoalkyl, sulfenatoaryl, etc. For example, azides (R—N₃) produce nitrenes (R—N:); diazoalkanes (R—CHN₂) produce carbenes (R—CH:); peroxides (RO—OR) produce alkoxy radicals (RO.); alkyl iodides (R-I) produce alkyl radicals (R.); and sulfenates (RS—OR) produce alkoxy radicals (RO.) and mercapto radicals (RS.). Alternatively, the reactive intermediates can be produced indirectly by exciting an aromatic photosensitizer; for example, Ar can transfer energy intramolecularly to an azide or other photoactive group and cause fragmentation.

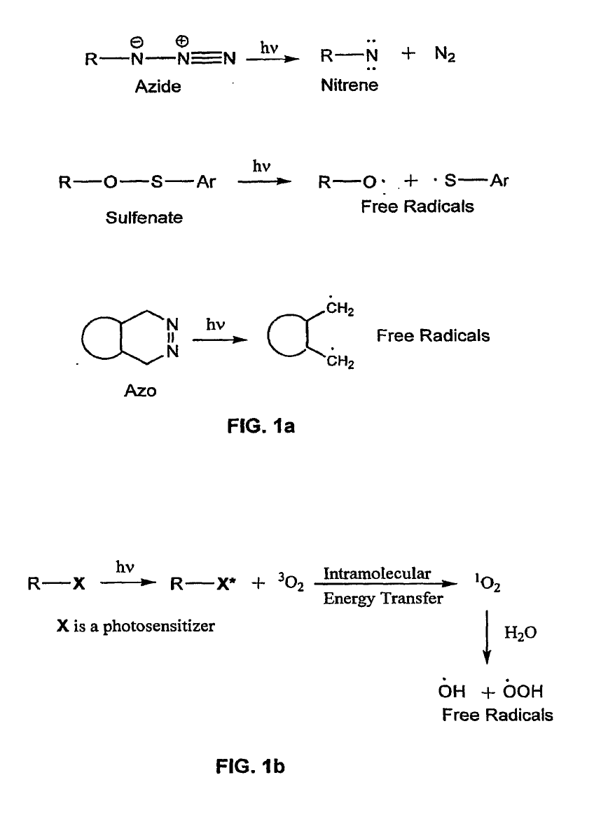

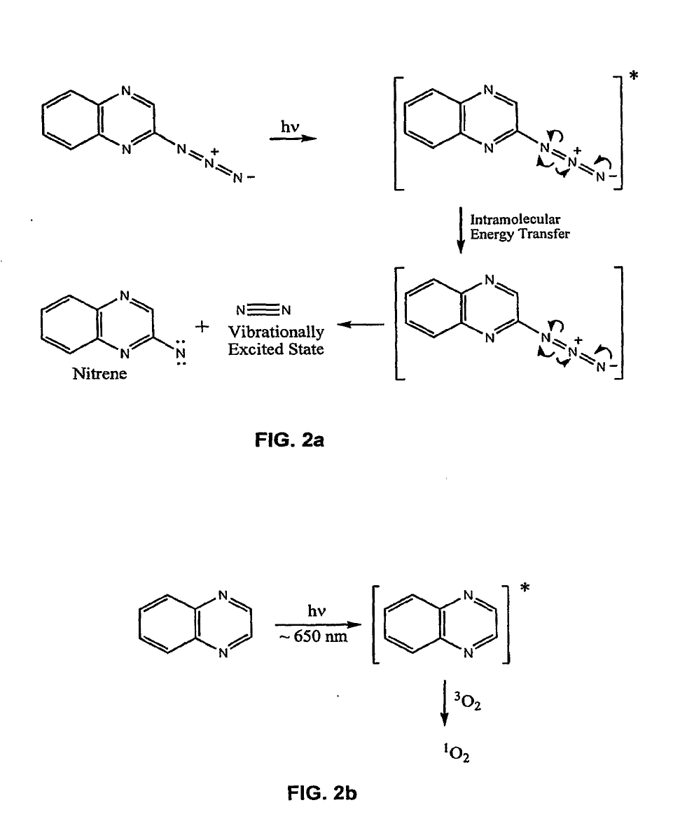

Photoactivation of photosensitizers of formulas I-VIII to produce nitrenes, renders such photosensitizers useful for Type 1 phototherapy, shown schematically in FIGS. 1 and 2A. Photoexcitation of Ar effects rapid intramolecular energy transfer to the azido group, resulting in bond rupture and production of nitrene and nitrogen gas. Photoexcitation of the aromatic photosensitizers effects rapid intramolecular energy transfer to the azide group, resulting in N—N bond rupture with concomitant extrusion of molecular nitrogen and formation of nitrene. The nitrogen that is released upon photofragmentation is in a vibrationally excited state that, upon relaxation, releases the energy to its surroundings in the form of heat that will result in tissue damage as well. Aliphatic azido compounds can also be used for phototherapy, but may require high-energy light for activation unless the azide moiety is attached to conjugated polyene system.

Photosensitizers of Formulas I-VIII may absorb in the red region of the electromagnetic spectrum and can transfer energy to oxygen molecules to generate singlet oxygen species. In some embodiments, photosensitizers of formulas I-VIII and bioconjugates thereof may be tuned to absorb in the red region and are, therefore, useful for Type 2 phototherapy.

The photosensitizers of Formulas I-VIII tend to have functional groups that absorb light in the visible region of the spectrum. They induce intramolecular energy transfer that results in photofragmentation of photoactive compounds such as azides, sulfenates, azo compounds, azidoalkyl, azidoaryl, diazoalkyl, diazoaryl, peroxoalkyl, peroxoaryl, iodoalkyl, azoalkyl, cyclic or acyclic azoalkyl, sulfenatoalkyl, sulfenatoaryl, etc. The photosensitizers of Formulas I-VIII are useful due to their small size and photophysical properties, in additional to their photochemical properties.

An exemplary embodiment of a compound of the invention that exhibits the general formula E1-L-Ar—X—PA is described below.

Ar is a photosensitizer selected from the Formulas I-VIII below;

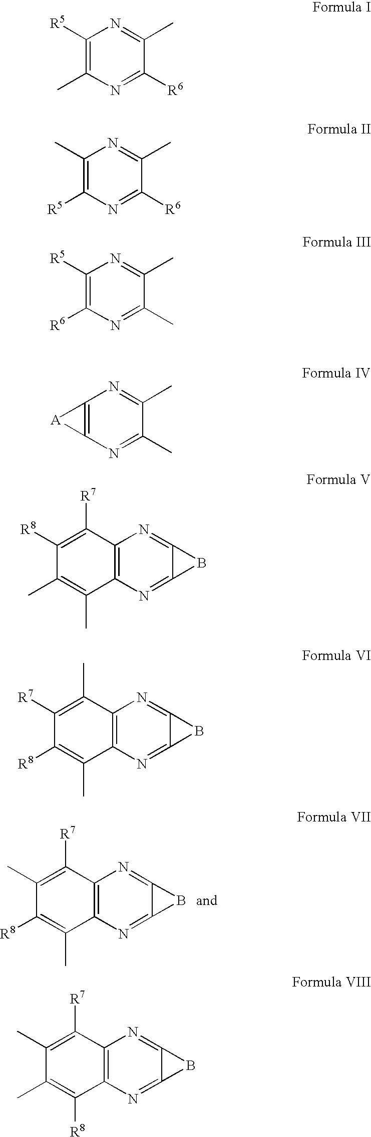

PA is selected from azide, azidoalkyl, azidoaryl, diazoalkyl, diazoaryl, peroxoalkyl, peroxoaryl, iodoalkyl, azoalkyl, cyclic or acyclic azoalkyl, sulfenatoalkyl, and sulfenatoaryl;

X, if present, is either a single bond or is selected from —(CH₂)ₐ—, —CO—OCO—, —HNCO—, —(CH₂)ₐCO—, —(CH₂)ₐOCO—, C₁-C₁₀ alkyl, C₅-C₁₀ aryl, C₅-C₁₀ heteroaryl, C₁-C₁₀ acyl, nitro, cyano, —(CH₂)ₐCO—, —(CH₂)ₐNR¹—, —NR¹CO—, —(CH₂)ₐCONR¹—, —(CH₂)ₐSO—, —(CH₂)ₐSO₂—, —(CH₂)ₐCON(R¹)—, —(CH₂)ₐN(R¹)CO—, —(CH₂)ₐN(R¹)CON(R²)— and —(CH₂)ₐN(R¹)CSN(R²)—;

L, if present, is a linker between the photosensitizer and the targeting moiety and is selected from —HNCO—, —CONR³, —(CH₂)_{b}—, —(CH₂)_{b}CONR³—, —N(R³)CO(CH₂)_{b}—, —OCO(CH₂)_{b}—, —(CH₂)_{b}CO₂—, —OCONH—, —OCO₂—, —HNCONH—, —HNCSNH—, —HNNHCO—, —OSO₂—, —NR³(CH₂)_{b}CONR⁴—, —CONR³(CH₂)_{b}NR⁴CO—, —NR³CO(CH₂)_{b}CONR⁴—, —(CH₂)_{b}CON(R³)—, —(CH₂)_{b}N(R³)CO—, —(CH₂)_{b}N(R³)CON(R⁴)—, and —(CH₂)_{b}N(R³)CSN(R⁴)—;

each of R¹ to R⁴ is independently selected from hydrogen, C1-C10 alkyl, —OH, C5-C10 aryl, C1-C10 hydroxyalky, C1-C10 polyhydroxyalkyl, C1-C10 alkoxyl, C1-C10 alkoxyalkyl, —SO₃H, —(CH₂)_{c}CO₂H, and —(CH₂)_{c}NR⁹R¹⁰;

each of R⁹ and R¹⁰ is independently selected from hydrogen, C1-C10 alkyl, C5-C10 aryl, and C1-C10 polyhydroxyalkyl;

each of a, b, and c independently ranges from 0 to 10.

each of A and B is independently selected from —(CH₂)_{d}Y(CH₂)ₑ, —C(R¹¹)═C(R¹²)—C(R¹³)═C(R¹⁴)—, —N═C(R¹²)—C(R¹³)═C(R¹⁴)—, —C(R¹¹)═N—C(R¹³)═C(R¹⁴)—, —C(R¹¹)═C(R¹²)—N═C(R¹⁴)—, —C(R¹¹)═C(R¹²)—C(R¹³)═N—, —C(R¹¹)═C(R¹²)—N(R¹⁵)—, —C(R¹¹)═C(R¹²)—O—, —C(R¹¹)═C(R¹²)—S—, —N═C(R¹¹)—N(R¹⁵)—, —N═C(R¹¹)—O—, —N═C(R¹¹)—S—, —C(R¹¹)═N—N(R¹⁵)—, —C(R¹¹)═N—N(R¹⁵)—, —C(R¹¹)═N—O—, —N═N—N(R¹⁵)—, —N═N—O— or —N═N—S—;

Y is selected from —O—, —NR¹⁶—, —S—, —SO— or —SO₂—, wherein each of d and e independently varies from 0 to 3, and R¹⁶ is selected from hydrogen, C₁-C₁₀ alkyl, C₅-C₁₀ aryl, C₁-C₁₀ hydroxyalkyl, or C₁-C₁₀ alkoxyalkyl;

wherein each of R⁵ to R⁸ and each of R¹¹ to R¹⁵ is independently selected from hydrogen, C₁-C₁₀ alkyl, C₅-C₁₀ aryl, C₁-C₁₀ hydroxyalkyl, C₁-C₁₀ alkoxyalkyl, C₅-C₁₀ heteroaryl, C₁-C₁₀ acyl, nitro, cyano, —(CH₂)_{f}N₃, —(CH₂)_{f}CO₂R¹⁶, —(CH₂)_{f}NR¹⁶R¹⁷, —NR¹⁶CON₃, —(CH₂)_{f}CONR¹⁶R¹⁷, —(CH₂)_{f}CON₃, —(CH₂)_{f}SON₃, —(CH₂)_{f}SO₂N₃, —(CH₂)_{f}CON(R¹⁶)E2, —(CH₂)_{f}N(R¹⁶)COE2, —(CH₂)_{f}N(R¹⁶)CON(R¹⁷)E2 or —(CH₂)_{f}N(R¹⁶)CSN(R¹⁷)E2, wherein f varies from 0 to 10 and each of R¹⁶ and R¹⁷ is independently selected from hydrogen, C₁-C₁₀ alkyl, C₅-C₁₀ aryl, C₁-C₁₀ hydroxyalkyl, or C₁-C₁₀ alkoxyalkyl; and each of E1 and E2 is independently hydrogen or a targeting moiety.

In some embodiments, each of E1 and E2, if present, is a whole or fragmented somatostatin receptor binding molecule, whole or fragmented ST receptor binding molecule, whole or fragmented neurotensin receptor binding molecule, whole or fragmented bombesin receptor binding molecule, whole or fragmented CCK receptor binding molecule, whole or fragmented steroid receptor binding molecule, or whole or fragmented carbohydrate receptor binding molecule.

In some embodiments, at least one of E1, R⁵ to R⁸, and R¹¹ to R¹⁵ is a targeting moiety where at least one of R⁵ to R⁸ or R¹¹ to R¹⁵ is selected from —(CH₂)_{f}CON(R¹⁶)E2, —(CH₂)_{f}N(R¹⁶)COE2, —(CH₂)_{f}N(R¹⁶)CON(R¹⁷)E2 and —(CH₂)_{f}N(R¹⁶)CSN(R¹⁷)E2. Further, f varies from 0 to 10, and each of R¹⁶ and R¹⁷ is independently selected from hydrogen, C₁-C₁₀ alkyl, C₅-C₁₀ aryl, C₁-C₁₀ hydroxyalkyl and C₁-C₁₀ alkoxyalkyl. The others substitutents are as previously defined.

The compound of the general formula may further comprise an electron donating group, an electron withdrawing group, a lipophilic group, and/or a hydrophilic group.

### Synthesis of photoactivator compounds, such as azido compounds, may be accomplished by a variety of methods known in the art, such as disclosed in S. R. Sandier and W. Karo, Azides. In Organic Functional Grouo Preparations (Second Edition), pp. 323-349, Academic Press: New York, 1986, which is expressly incorporated by reference herein in its entirety. Aromatic azides derived from acridone, xanthone, anthraquinone, phenanthridine, and tetrafluorophenyl systems have been shown to photolyze in the visible and in UV-A regions, for example, L. K. Dyall and J. A. Ferguson, Pyrolysis of aryl azides. XI Enhanced neighbouring group effects of carbonyl in a locked conformation. Australian Journal of Chemistry, 1992, 45, 1991-2002; A. Y. Kolendo, Unusual product in the photolysate of 2-azidoxanthone. Chemistry of Heterocyclic Compounds, 1998, 34(10), 1216; R. Theiler, Effect of infrared and visible light on 2-azidoanthraquinone in the QA binding site of photosynthetic reaction centers. An unusual mode of activation of photoaffinity label. Biological Chemistry Hoppe-Seyler, 1986, 367(12), 1197-207; C. E. Cantrell and K. L. Yielding, Repair synthesis in human lymphocytes provoked by photolysis of ethidium azide. Photochemistry and Photobiology, 1977, 25(2), 1889191; and R. S. Pandurangi et al., Chemistry of bifunctional photoprobes 3: correlation between the efficiency of CH insertion by photolabile chelating agents. First example of photochemical attachment of 99mTc complex with human serum albumin. Journal of Organic Chemistry, 1998, 63, 9019-9030, each of which is expressly incorporated by reference herein in its entirety. The compounds may contain additional functionalities that can be used to attach various types of biomolecules, synthetic polymers, and organized aggregates for selective delivery to various organs or tissues of interest. Examples of synthetic polymers include polyaminoacids, polyols, polyamines, polyacids, oligonucleotides, aborols, dendrimers, and aptamers.

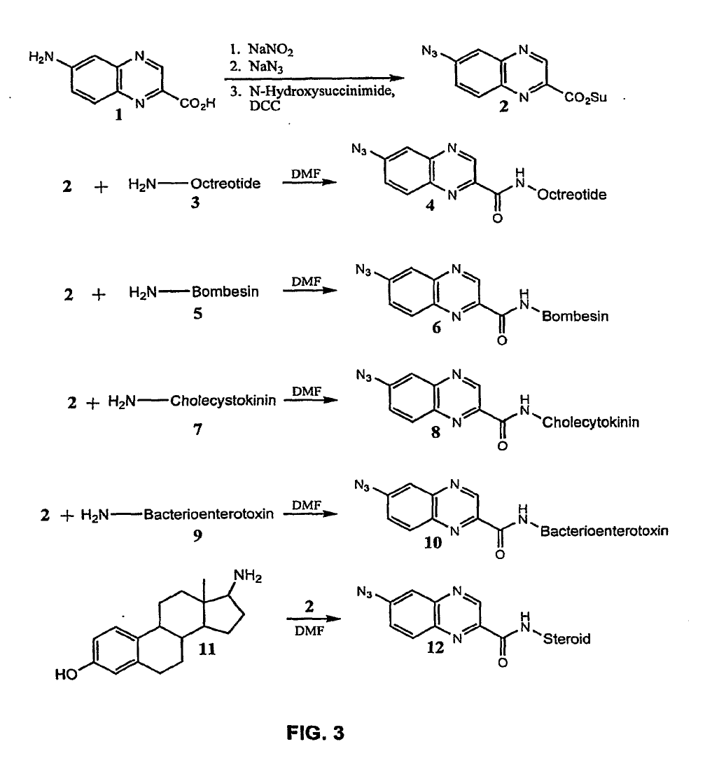

The general synthesis of compounds of the type shown in formulas I-VIII has been known for several decades, and can be readily prepared by the methods well known in the art. See: The Pyrazines. The Chemistry of Heterocyclic Compounds, G. B. Barlin, Ed., J. Wiley, New York, 1982; and The Pyrazines: Supplement 1. The Chemistry of heterocyclic compounds, D. J. Brown, Ed., J. Wiley, New York, 2002. The coupling of biomolecules such as somatostatin, bombesin, cholecystokinin, bacternoenterotoxin, steroids, and the like to compounds of formulas I-VIII can be achieved by the use of succinimido active esters, for example, as illustrated in FIG. 3.

In one example, the targeting moiety of the inventive compound may contain all or part of a steroid hormone or a steroid receptor binding compound, and therefore target steroid hormone sensitive receptors. In this example, the compound is administered, targets the desired site such as a lesion of the breast and/or prostate, is photoactivated, and forms free radicals at this site thereby effecting cell injury or death at the desired target site. Similar target binding compounds and uses will be recognized by one skilled in the art. For example, the targeting moiety may be a compound that targets and binds to a somatostatin, bombesin, CCK, and/or neurotensin receptor binding molecule, or may be a carcinogenic embryonic antigen-binding compound that binds to a carcinogenic embryonic antigen. These are then photoactivated for radical formation at, for example, lung cancer cells with CCK receptor binding compounds, colorectal cancer cells with ST receptor and carcinoembryonic antigen (CEA) binding compounds, melanoma cells with dihyroxyindolecarboxylic acid, vascular sites of atherosclerotic plaque with integrin receptor binding compounds, brain lesions with amyloid plaque binding molecules, etc.

Successful specific targeting of fluorescent dyes to tumors using antibodies and peptides for diagnostic imaging of tumors has been demonstrated by us and others as described in Achilefu et al., Novel receptor-targeted fluorescent contrast agents for in vivo imaging of tumors, Investigative Radiology, 2000, 35, pp. 479-485; Ballou et al., Tumor labeling in vivo using cyanine conjugated monoclonal antibodies, Cancer Immunology and Immunotherapy, 1995, 41, pp. 257-263; and Licha et al., New contrast agent for optical imaging: acid cleavable conjugates of cyanine dyes with biomolecules, in Biomedical Imaging: Reporters, Dyes and Instrumentation, Proceedings of SPIE, 1999, 3600, pp. 2935, each of which is expressly incorporated by reference herein in its entirety. Therefore, receptor-targeted photochemicals are effective in reaching and activation at the site of various lesions.

Some exemplary methods of performing photochemical procedures using compounds including photosensitizers of formulas I-VIII encompass administering to a patient an effective amount of a compound of the invention in a biologically acceptable formulation. The compound is activated, either immediately or after allowing an interval for its accumulation at a target site, followed by illumination with light of wavelength 300 to 1200 nm, preferably 350 to 850 nm, at the site of the lesion. If the lesion is on the skin surface, or on a photo-accessible surface other than skin, such as a mucosal surface of the oral cavity, vagina, or nasal cavity, it may be directly illuminated. If the lesion is in or on a cavity, it may be illuminated with an endoscopic catheters equipped with a light source. Such an application may be used, for example, with a lesion in a blood vessel, lung, heart, throat, ear, rectum, bladder, stomach, intestines, or esophagus. For a lesion in an organ, such as liver, brain, prostate, breast, pancreas, etc., a photochemical compound in the tissue can be illuminated using a surgical instrument (forceps, scalpel, etc.) containing or configured with an illumination system. Such instruments are known to one skilled in the art, such as fiber optic instruments available from BioSpec (Moscow, 11991, Russia) for example, TC-I fiber optic tool for photodynamic therapy with fine needle tip for irradiating interstitial tumors. A surgeon performing a procedure is thus able to expose a tumor or other target tissue to light of a desired wavelength, power, and fluence rate during a procedure. The intensity, power, duration of illumination, and the wavelength of the light may vary widely depending on the location and site of the lesions. The fluence rate is preferably, but not always, kept below 200 mW/cm² to minimize thermal effects. Appropriate power depends on the size, depth, and pathology of the lesion. The inventive compounds have broad clinical utility that includes, but is not limited to, phototherapy of tumors, inflammatory processes, and impaired vasculature.

The particular wavelength(s) required for photoactivation to achieve phototherapy with a specific compound may be determined in a variety of ways. As one example, it may be determined empirically from exposing the synthesized compound to light of varying wavelength and thereafter assaying to determine the extent of tissue damage at a targeted site. It may also be determined based upon the known photoactivation maxima for the particular photosensitizer. In general, agents that act via a Type 1 mechanism can be activated across a wide wavelength spectrum from about 300 nm to about 950 nm. Thus, activation of a Type 1 component or compound may be achieved using an activation wavelength in this range.

Exemplary compositions of the invention can be formulated for enteral (oral or rectal), parenteral, topical, or cutaneous administration. A formulation may be prepared using any of the compounds previously described, along with excipients, buffers, etc., to provide a composition for administration by any one of a variety of routes. Compositions of the invention may be injected, ingested, applied topically, transdermally, subcutaneously, administered by aerosol formulation and/or inhalation, etc. After administration, a composition accumulates, for example, at a target tissue if a targeting moiety is included in the compound. The selected target site, or a site requiring diagnosis or treatment, is exposed to light with a sufficient power and fluence rate to render a diagnosis and/or treatment. Topical or cutaneous delivery may include aerosols, creams, gels, solutions, etc. Compositions of the invention are administered in doses effective to achieve the desired objective. Such doses may vary widely depending upon the particular complex employed, the organs or tissues to be examined, the equipment employed in the clinical procedure, the efficacy of the treatment achieved, and the like. Compositions of the invention can contain an effective amount of the phototherapeutic agent along with conventional pharmaceutical carriers and excipients appropriate for the type of administration contemplated. Such compositions may include stabilizing agents and skin penetration enhancing agents and/or also contain pharmaceutically acceptable buffers, emulsifiers, surfactants, and, optionally, electrolytes such as sodium chloride.

Formulations for enteral administration may vary widely as is well known in the art. In general, such formulations are liquids, which include an effective amount of the composition in an aqueous solution or suspension. Such enteral compositions may optionally include buffers, surfactants, emulsifiers, thixotropic agents, and/or the like. Compositions for oral administration may also contain flavoring agents and other ingredients for enhancing their organoleptic qualities. A topical application can be formulated as a liquid solution, water/oil emulsion, or suspension of particles, depending on the particular nature of the agent and the type of tissue to be targeted. The compositions may also be delivered in an aerosol spray.

If an inventive compound is water soluble, for example, a solution in water may be applied to or into the target tissue. Delivery into and through the skin may be enhanced by using well known methods and agents such as transdermal permeation enhancers, for example, “azone”, N-alkylcyclic amides, dimethylsulfoxide, long-chained aliphatic acids (C₁₀), etc. If an inventive compound is not water soluble, it may be dissolved in a biocompatible oil (e.g. soybean oil, fish oil, vitamin E, linseed oil, vegetable oil, glyceride esters, and/or long-chained fatty esters) and emulsified with surface-active compounds (e.g. vegetable or animal phospholipids; lecithin; long-chained fatty salts and alcohols; Pluronics: polyethylene glycol esters and ethers; etc.) in water to make a topical cream, suspension, water/oil emulsion, water/oil microemulsion, or liposomal suspension to be delivered or applied to the target region. In the case of liposomes, an inventive compound may be attached to or be contained in the lamellar material.

The dose of compound may vary from about 0.1 mg/kg body weight to about 500 mg/kg body weight. In one embodiment, the dose is in the range of about 0.5 mg/kg body weight to about 2 mg/kg body weight. As one example, for compositions administered parenterally, a sterile aqueous solution or suspension of compound may be present in a concentration ranging from about 1 nM to about 0.5 M, typically in a concentration from about 1 μM to about 10 mM.

In general, a formulated compound including at least one photosensitizer of Formulas I-VIIII is administered at a dose or in a concentration that is effective, upon exposure to light, to generate radicals at a target tissue such that cells at the target tissue are injured or killed. The target tissue is exposed for a period of time to light of a wavelength that is effective to activate the compound that produces Type 1 destruction in the target tissue. In the case of ex vivo or in vitro use (e.g., tissue culture), a formulated compound including at least one photosensitizer of Formulas I-VIII is administered at a dose or in a concentration that is effective, upon exposure to light, to generate radicals within a biological medium (e.g., culture medium or organ preservation fluid) such that target tissue in the biological medium are injured or killed. The biological medium is exposed for a period of time to light of a wavelength that is effective to activate the compound that produces Type 1 destruction in the target tissue.

The concentration of an inventive compound at the target tissue is the outcome of either passive or active uptake processes in the tissue. An example of passive uptake would be where the compound is attached or is contained within a particulate carrier. If the carrier is of an appropriate size, in the range of about 100 nm to about 1000 nm, it will leak into the perfusion boundary of vascular tumors. An example of active uptake would be where a receptor based attachment binds a particular receptor that is expressed on the target tissue. The effective concentration of a compound of the invention thus depends on the nature of the formulation, method of delivery, target tissue, activation method and toxicity to the surrounding normal tissue. Formulations for topical delivery may also contain liquid or semisolid excipients to assist in the penetration of the photosensitizer.

In some embodiments, compositions of the invention may be formulated as micelles, liposomes, microcapsules, microparticles, nanocapsules, nanoparticles, or the like. These formulations may enhance delivery, localization, target specificity, administration, etc. As one example, a liposome formulation of an inventive compound may be beneficial when the compound does not contain a specific targeting moiety (e.g., when E is hydrogen). As another example, a liposome formulation of an inventive compound may be beneficial when the compound has solubility limitations. Preparation and loading of these are well known in the art.

As one example, liposomes may be prepared from dipalmitoyl phosphatidylcholine (DPPC) or egg phosphatidylcholine (PC) because this lipid has a low heat transition. Liposomes are made using standard procedures as known to one skilled in the art (e.g., Braun-Falco et al., (Eds.), Griesbach Conference, Liposome Dermatics, Springer-Verlag, Berlin (1992)). Polycaprolactone, poly(glycolic) acid, poly(lactic) acid, polyanhydride or lipids may be formulated as microspheres. As an illustrative example, the optical agent may be mixed with polyvinyl alcohol (PVA), the mixture then dried and coated with ethylene vinyl acetate, then cooled again with PVA. In a liposome, the optical agent may be within one or both lipid bilayers, in the aqueous between the bilayers, or with the center or core. Liposomes may be modified with other molecules and lipids to form a cationic liposome. Liposomes may also be modified with lipids to render their surface more hydrophilic which increases their circulation time in the bloodstream. The thus-modified liposome has been termed a “stealth” liposome, or a long-lived liposome, as described in U.S. Pat. Nos. 6,277,403; 6,610,322; 5,631,018; 5,395,619; and 6,258,378, each of which is expressly incorporated by reference herein in its entirety, and in Stealth Liposomes, Lasic and Martin (Eds.) 1995, CRC Press, London, specifically pages 1-6, 13-62, 93-126, 139-148, 197-210, and 233-244. Encapsulation methods include detergent dialysis, freeze drying, film forming, injection, as known to one skilled in the art and disclosed in, for example, U.S. Pat. No. 6,406,713 which is expressly incorporated by reference herein in its entirety.

A compound including at least one photosensitizer of Formulas I-VIII formulated in liposomes, microcapsules, etc. may be administered by any of the routes previously described. In a formulation applied topically, the optical agent may be slowly released over time. In an injectable formulation, the liposome capsule may circulate in the bloodstream and to be delivered to a desired site. The use of liposomes, microcapsules, or other microparticles allows the incorporation of two or more inventive compounds of different types and capabilities in a single, inventive composition.

A compound of the invention containing at least one photosensitizer of Formulas I-VIII could be also used as an antimicrobial agent and used for the treatment of infections, wounds, and/or burn healing, as described by Hamblin et al., in “Targeted photodynamic therapy for infected wounds in mice” in Optical Methods for Tumor Treatment and Detection: Mechanisms and Techniques in Photodynamic Therapv XI (Proceedings of SPIE 2002) which is expressly incorporated by reference herein in its entirety. In this regard, the use of liposomes etc., as delivery vehicles for compounds of the invention would be desired. For example, a compound of the invention may be partially or totally encapsulated in a liposome or other microparticle. E may be hydrogen or a targeting moiety as previously described. The encapsulated compound may be administered to a patient whereby it may localize at an infected site. A photochemical procedure performed to detect the compound at the infected site and subsequently treat the infected area by activating the compound to kill the infectious agent.

The following example illustrates a specific embodiment of the invention pertaining to the preparation and properties of a compound of the invention derived from bombesin (a bioactive peptide) and a photochemical compound.

### EXAMPLE

### Synthesis of Photochemical Compound-Bombesin (7-14) Conjugate

The peptide is prepared by fluorenylmethoxycarbonyl (Fmoc) solid phase peptide synthesis strategy with a commercial peptide synthesizer from Applied Biosystems (Model 432A SYNERGY Peptide Synthesizer). The first peptide cartridge contains Wang resin pre-loaded with an amide resin on 25-mole scale. The amino acid cartridges are placed on the peptide synthesizer, and the product is synthesized from the C— to the N-terminal position. Coupling of the Fmoc-protected amino acids (75 μmol) to the resin-bound free terminal amine (25 μmol) is carried out with 2-(1H-benzotriazol-1-yl)-1,1,3,3-tetramethyluronium hexafluorophosphate (HBTU, 75 μmol)/N-hydroxybenzotriazole (HOBt, 75 μmol). Each Fmoc protecting group on solid support is removed with 20% piperidine in dimethylformamide before the subsequent amino acid is coupled to it. The last cartridge contains the Ar—PA compound, which is coupled to the peptide automatically, thus avoiding the need for post-synthetic manipulations.

After the synthesis is completed, the product is cleaved from the solid support with a cleavage mixture containing trifluoroacetic acid (85%):water (5%):phenol (5%):thioanisole (5%) for six hours. The peptide-photosensitizer/photoactive compound conjugate is precipitated with t-butyl methyl ether and lyophilized in water:acetonitrile (2:3) mixture. The conjugate is purified by HPLC and analyzed with LC/MS.

It should be understood that the embodiments of the present invention shown and described in the specification are only exemplary embodiments of the invention and are not limiting in any way. As known to one skilled in the art, various changes and modifications are possible and are contemplated within the scope of the invention described. For example, compounds containing polycyclic aromatic photosensitizers may also be used in optical diagnostic imaging. Therefore, various changes, modifications or alterations to those embodiments may be made or resorted to without departing from the spirit of the invention and the scope of the following claims.

What is claimed is:

1-17. (canceled)

18. A method of using a compound, the method comprising:
administering an effective amount of a compound to an animal; and
exposing the administered compound to light sufficient to activate the compound, wherein the compound is of the formula E1-L-Ar—X—PA, and wherein:
Ar is selected from 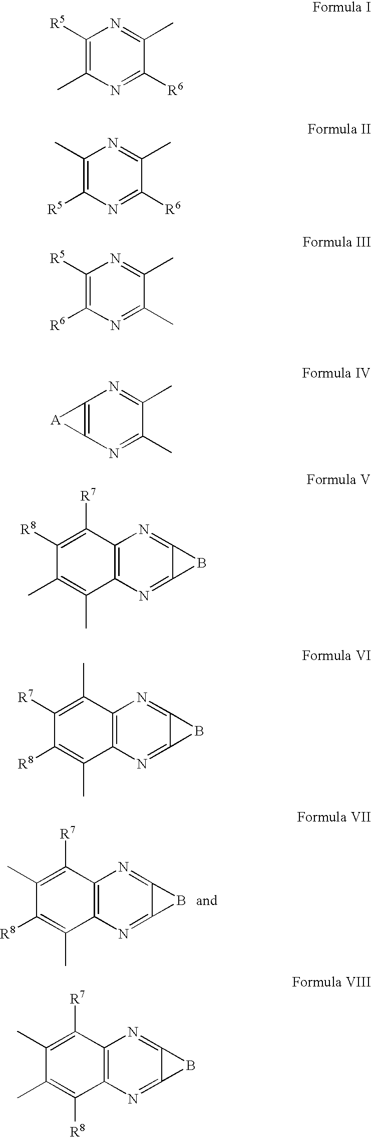
PA is selected from azide, azidoalkyl, azidoaryl, diazoalkyl, diazoaryl, peroxoalkyl, peroxoaryl, iodoalkyl, azoalkyl, cyclic or acyclic azoalkyl, sulfenatoalkyl, sulfenatoaryl, and combinations thereof;
X, if present, is either a single bond or is selected from —(CH₂)ₐ—, —CO—OCO—, —HNCO—, —(CH₂)ₐCO—, —(CH₂)ₐOCO—, C₁-C₁₀ alkyl, C₅-C₁₀ aryl, C₅-C₁₀ heteroaryl, C₁-C₁₀ acyl, nitro, cyano, —(CH₂)ₐCO₂—, —(CH₂)ₐNR¹—, —NR¹CO—, —(CH₂)ₐCONR¹—, —(CH₂)ₐSO—, —(CH₂)ₐSO₂—, —(CH₂)ₐCON(R¹)—, —(CH₂)ₐN(R¹)CO—, —(CH₂)ₐN(R¹)CON(R²)— and —(CH₂)ₐN(R¹)CSN(R²)—;
L, if present, is selected from —HNCO—, —CONR³, —(CH₂)_{b}—, —(CH₂)_{b}CONR³—, —N(R³)CO(CH₂)_{b}—, —OCO(CH₂)_{b}—, —(CH₂)_{b}CO₂—, —OCONH—, —OCO₂—, —HNCONH—, —HNCSNH—, —HNNHCO—, —OSO₂—, —NR³(CH₂)_{b}CONR⁴—, —CONR³(CH₂)_{b}NR⁴CO—, —NR³CO(CH₂)_{b}CONR⁴—, —(CH₂)_{b}CON(R³)—, —(CH₂)_{b}N(R³)CO—, —(CH₂)_{b}N(R³)CON(R⁴)— and —(CH₂)_{b}N(R³)CSN(R⁴)—;
each of R¹ to R⁴ is independently selected from hydrogen, C1-C10 alkyl, —OH, C5-C10 aryl, C1-d10 hydroxyalky, C1-C10 polyhydroxyalkyl, C1-C10 alkoxyl, C1-C10 alkoxyalkyl, —SO₃H, —(CH₂)_{c}CO₂H, and —(CH₂)_{c}NR⁹R¹⁰;
each of R⁹ and R¹⁰ is independently selected from hydrogen, C1-C10 alkyl, C5-C10 aryl, and C1-C10 polyhydroxyalkyl;
each of a, b, and c independently ranges from 0 to 10;
each of A and B is independently selected from —(CH₂)_{d}Y(CH₂)ₑ—, —C(R¹¹)═C(R¹²)—C(R¹³)═C(R¹⁴)—, —N═C(R¹²)—C(R¹³)═C(R¹⁴)—, —C(R¹¹)═N—C(R¹³)═C(R¹⁴)—, —C(R¹¹)═C(R¹²)—N═C(R¹⁴)—, —C(R¹¹)═C(R¹²)—C(R¹³)═N—, —C(R¹¹)═C(R¹²)—N(R⁵)—, —C(R¹¹)═C(R¹²)—O—, —C(R¹¹)═C(R¹²)—S—, —N═C(R¹¹)—N(R¹⁵)—, —N═C(R¹¹)—O—, —N═C(R¹¹)—S—, —C(R¹¹)═N—N(R¹⁵)—, —C(R¹¹)═N—N(R¹⁵)—, —C(R¹¹)═N—O—, —N═N—N(R¹⁵)— and —N═N—O— or —N═N—S—;
Y is selected from —O—, —NR¹⁶—, —S—, —O— and —SO₂—;
each of d and e independently varies from 0 to 3;
R¹⁶ is selected from hydrogen, C₁-C₁₀ alkyl, C₅-C₁₀ aryl, C₁-C₁₀ hydroxyalkyl, and C₁-C₁₀ alkoxyalkyl;
each of R⁵ to R⁸ and each of R¹¹ to R¹⁵ is independently selected from hydrogen, C₁-C₁₀ alkyl, C₅-C₁₀ aryl, C₁-C₁₀ hydroxyalkyl, C₁-C₁₀ alkoxyalkyl, C₅-C₁₀ heteroaryl, C₁-C₁₀ acyl, nitro, cyano, —(CH₂)_{f}N₃, —(CH₂)_{f}CO₂R¹⁶, —(CH₂)_{f}R¹⁶R¹⁷, —NR¹⁶CON₃, —(CH₂)_{f}CONR¹⁶R¹⁷, —(CH₂)_{f}CON₃, —(CH₂)_{f}SON₃, —(CH₂)_{f}SO₂N₃, —(CH₂)_{f}CON(R¹⁶)E2, —(CH₂)_{f}N(R¹⁶)COE2, —(CH₂)_{f}N(R¹⁶)CON(R¹⁷)E2, and —(CH₂)_{f}N(R¹⁶)CSN(R¹⁷)E2, wherein f varies from 0 to 10, and each of R¹⁶ and R¹⁷ is independently selected from hydrogen, C₁-C₁₀ alkyl, C₅-C₁₀ aryl, C₁-C₁₀ hydroxyalkyl, and C₁-C₁₀ alkoxyalkyl; and
each E1 and E2 is independently hydrogen or a targeting moiety.

19. The method of claim 18, wherein each E1 and E2, if present, is selected from whole or fragmented somatostatin receptor binding molecules, whole or fragmented ST receptor binding molecules, whole or fragmented neurotensin receptor binding molecules, whole or fragmented bombesin receptor binding molecules, whole or fragmented cholecystekinin (CCK) receptor binding molecules, whole or fragmented steroid receptor binding molecules, and whole or fragmented carbohydrate receptor binding molecules.

20. The method of claim 18, further comprising:
allowing the compound to accumulate in a target tissue of the animal before the exposing.

21. The method of claim 18 resulting in Type 1 therapy, Type 2 therapy, or a combination of Types 1 and 2 therapy.

22. The method claim 18, wherein a reactive intermediate results by exciting the Ar substituent of the compound to transfer energy intramolecularly to the PA substituent of the compound.

23. The method of claim 18, wherein the light to which the administered compound is exposed is between about 300 nm and about 950 nm.

24. The method of claim 18, resulting in a necrotic effect, an antimicrobial effect, an apoptotic effect, or a combination thereof.

25. The method of claim 18, wherein the administering comprises administering a biocompatible composition to the animal, wherein the biocompatible composition comprises an effective amount of the compound and at least one biocompatible excipient.

26. The method of claim 25, wherein the at least one biocompatible excipient comprises a buffer, emulsifier, surfactant, electrolyte, or combination thereof.

27. The method of claim 25, wherein the biocompatible composition comprises liposomes, micelles, microcapsules, microparticles, or a combination thereof that include the compound.

28. The method of claim 25, wherein the composition is administered in a range of about 0.1 mg/kg body weight to about 500 mg/kg body weight.

29. The method of claim 25, wherein the composition is administered in a range of about 0.5 mg/kg body weight to about 2 mg/kg body weight.

30. The method of claim 25, wherein the composition is parenterally administered in a concentration range of 1 nM to 0.5 M.

31. The method of claim 25, wherein the composition is administered by a route selected from parenteral, enteral, topical, aerosol, subdermal, subcutaneous, inhalation, and combinations thereof.

32. The method of claim 25, wherein the composition is administered in a form selected from an aerosol spray, a cream, a gel, and a solution.
